(19) United States
(12) Patent Application Publication
(10) Pub. No.: US 2019/0365324 A1
(43) Pub. Date: Dec. 5, 2019

(54) ACTIVE DEVICE FOR MONITORING ANIMAL

(71) Applicant: KONYANE INJECTION MOLD CO., LTD, New Taipei City (TW)

(72) Inventor: I Hsien CHANG, New Taipei City (TW)

(21) Appl. No.: 15/992,291

(22) Filed: May 30, 2018

(51) Int. Cl. A61B 5/00; A61B 5/0205; A01K 11/00

(52) U.S. Cl. CPC A61B 5/6839; A61B 5/0008; A61B 5/02055; A61B 5/01; A61B 5/6815; A61B 5/7445; A01K 11/004; A61B 5/0022

(57) ABSTRACT

Disclosed here is an active device for monitoring animal being capable of measuring vital signs of the animal and actively signaling an external apparatus. The device comprises a pin adapted to penetrate a predetermined portion of the animal; a gasket having two ring portions formed as one piece; a base having a first sleeve portion for allowing the pin to squeeze through so as to be engaged with each other and blocking the pin from slipping off after engagement; a monitoring unit; and a cover having a second sleeve portion to receive the first sleeve portion, and being joined with the base in a waterproof manner so as to accommodate the monitoring unit therein.

### FIELD OF THE INVENTION

The field of invention relates generally to an active device for monitoring animal, and, in particular to a device capable of measuring, and actively and wirelessly transmitting vital signs of animal, such as body temperature, heart rate, blood pressure, etc.

### BACKGROUND OF THE INVENTION

Since vital signs such as body temperature, heart rate, blood pressure, etc. are important data in judging animal health status, and therefore are used as key references to take necessary measures such as medical treatments and isolation, vital signs of fed animals of high economic value like pig, cow, sheep, horse, etc. need to be measured and monitored for avoiding or decreasing losses.

A variety of electronic devices have been applied to monitor animals. For example, passive electronic tags may be attached to bodies of animals for purpose of identity, and another electronic device may be mounted on an animal to monitor its location, body temperature, and so on. For purpose of monitoring vital signs such as body temperature, a monitoring device may be mounted on at least one portion in an animal body, and for purpose of accurate measurement, a fastening design adapted to securely fastening the monitoring device onto the animal body should depend on the portion of animal body to be mounted with the device.

Therefore, for considerable reduction of feeding cost, it is desired that an active monitoring device can be securely fastened onto an appropriate portion of an animal body to accurately measure vital signs such as body temperature, heart beat, etc., and to actively report the measured data during a feeding period.

### SUMMARY OF THE INVENTION

According to an embodiment of the invention, an active device for monitoring animal being capable of measuring vital signs of the animal and actively signaling an external apparatus comprises a pin adapted to penetrate a predetermined portion of the animal; a gasket having two ring portions formed as one piece; a base having a first sleeve portion for allowing the pin to squeeze through so as to be engaged with each other and blocking the pin from slipping off after engagement; a monitoring unit; and a cover having a second sleeve portion to receive the first sleeve portion, and being joined with the base in a waterproof manner so as to accommodate the monitoring unit therein. Preferably, the animal is a livestock.

Preferably, the monitoring unit includes a sensor module for measuring vital signs of the animal; a wireless communication module adapted to communicate with the external apparatus; a controller adapted to receive measured data from the sensor module, and to control the wireless communication module to transmit the measured data to the external apparatus; and a battery for powering the active device. The sensor module comprises at least one sensor selected from a temperature sensor, a blood pressure sensor, a heart rate sensor, and an accelerator sensor.

Further, the wireless communication module uses at least one of Lora, or Zigbee wireless communication standards to communicate with the external apparatus.

Preferably, the monitoring unit further comprises an LED indicator adapted to display a status of the device, and when the controller detects that electricity of the battery is low, the LED indicator is caused to indicate a low power status.

The active device for monitoring animal of this invention can be easily and reliably fastened into ears of animals, automatically and accurately measure vital signs of animals like body temperature, and actively report the measured data, resulting in elimination or reduction of mass infection or mass mortality of fed animals, and considerable reduction of feeding cost.

### BRIEF DESCRIPTION OF THE DRAWINGS

In the following description, various embodiments are described with reference to the following drawings. In the drawings, like reference characters generally refer to the same parts throughout the different views. The drawings are not necessarily to scale, emphasis instead generally being placed upon illustrating the principles of the invention.

FIG. 1 illustrates the appearance of the active device for monitoring animal according to embodiments of this invention.

FIG. 2 is an explosion view of the active device for monitoring animal according to embodiments of this invention.

FIG. 3 is an enlarged view showing a base of the active device for monitoring animal according to embodiments of this invention.

FIG. 4 is a block diagram illustrating a monitoring unit according to embodiments of this invention.

### DETAILED DESCRIPTION OF THE PREFERRED EMBODIMENTS

In the following description, numerous specific details are set forth to aid in full understanding of discussed implementations. However, it is understood that embodiments of the invention may be practiced without these specific details. In other instances, well-known circuits, structures and techniques have been omitted or simplified in order not to obscure understanding of this description.

In a livestock farm where a plurality of animals are fed, it is required to continuously measure and report vital signs of livestock such as body temperature so as to cure livestock in time or prevent mass mortality. In general, measurement accuracy and fastness should be taken into account if a monitoring device is mounted onto an animal. In view of these, an active device for monitoring animal according to embodiments of this invention is preferably mounted in an ear of the animal. The active monitoring device according to embodiments of this invention is capable of wireless transmit measured data to external communication apparatus such as gateways and routers. Besides, it is required that the active monitoring device can normally operate during the feeding period such as 3 years, without replacing a battery for powering the device.

The structure of the active device for monitoring animal according to embodiments of this invention will be explained in the following description.

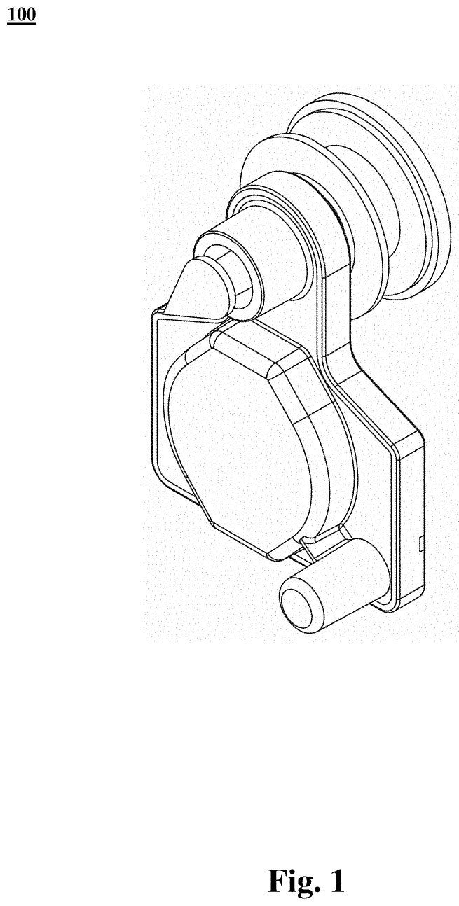

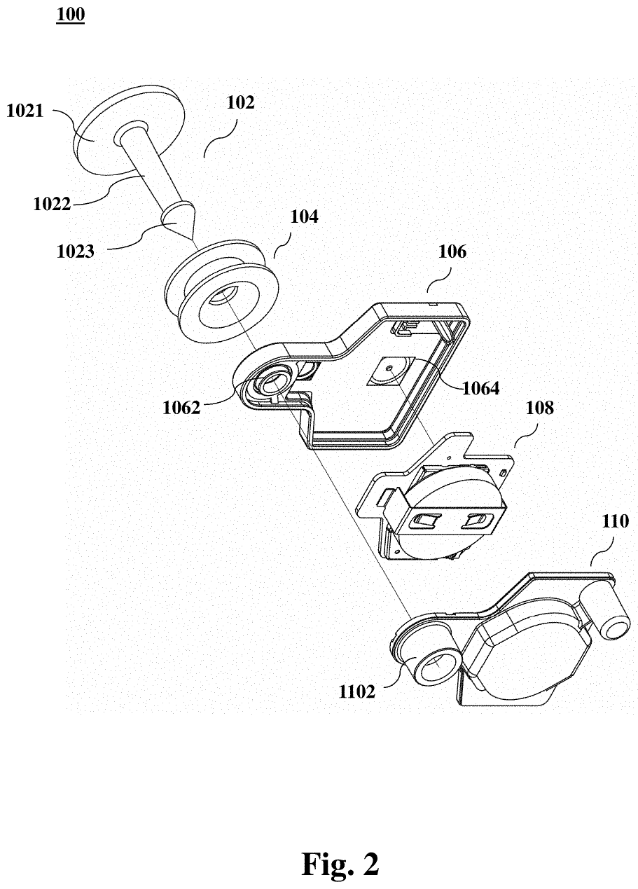

First, the active device for monitoring animal according to embodiments of this invention will be discussed with reference to FIGS. 1 and 2. FIG. 1 illustrates the appearance of the active device 100 for monitoring animal according to embodiments of this invention. FIG. 2 is an explosion view of the active device 100. As illustrated in FIG. 2, the active device 100 for monitoring animal includes a pin 102, a gasket 104, a base 106, a monitoring unit 108, and a cover 110.

For example, the pin 102 is formed with a larger head portion 1021, a post 1022 projected from the head portion 1021, and a cone tip 1023 formed on one end of the post 1022. According to embodiments, the cone tip 1023 is made of metal or alloy so as to easily penetrate a portion of the animal body such as ears, and the other portions of the pin 102 are made of plastic, resin, or any other material different from metal. Alternatively, the whole pin 102 may be made of metal or alloy. The pin 102 is not limited to this, but may be formed with any other constructions or forms, which can penetrate a predetermined portion of animal body and then can be coupled with a corresponding portion of the base 106.

According to embodiments, the gasket 104, for example, may be made of resin, rubber, plastic, silica gel, and so on. Here, the gasket 104 is a double-ring type, in which two o-ring portions are joined by a hollow tube so as form the one-piece gasket. Since measurement performance of sensors (described later) mounted in the monitoring unit 108 is easily influenced by the distance between the measured portion of animal body and the sensors, especially in measuring the body temperature, the gasket 104 is used to make the whole device 100 more securely fastened into the portion of animal body such as ears, and to ensure the distance between the sensors and the measured portion to be maintained in a predetermined range, resulting a significant improvement to measurement performance of the vital signs such as body temperature.

The base 106, the monitoring unit 108, and the cover 110 constitute the main body of the active device 100 for monitoring animal.

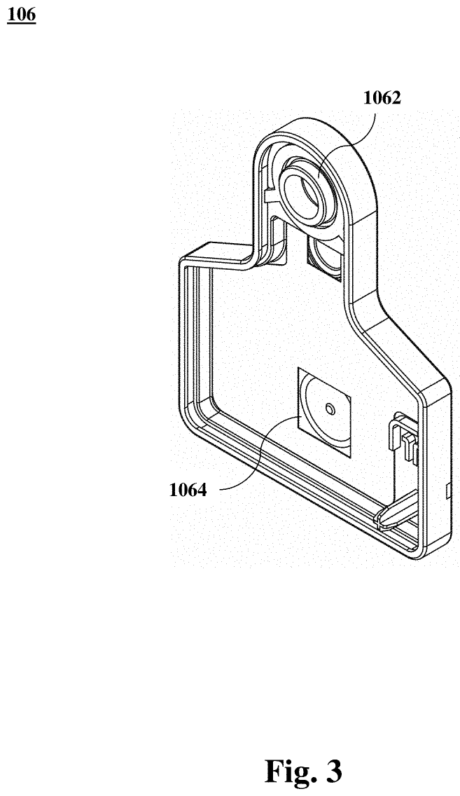

Below, the base 106 will be illustrated with reference to FIGS. 2 and 3. An inner sleeve portion 1062 is formed in the top of the base 106. The outer diameter of the inner sleeve portion 1062 is smaller than the inner diameter of an outer sleeve portion 1102 formed in the top of the cover 110. Therefore, the inner sleeve portion 1062 can be fit to be inserted into the outer sleeve portion 1102, when the base 106 and the cover 110 are engaged with each other. It is noted that the inner sleeve portion 1062 and the outer sleeve portion 1102 are made of materials having different hardness. In specific, the material forming the inner sleeve portion 1062 has a hardness lower than that forming the outer sleeve portion 1102. Further, the inner diameter of the inner sleeve portion is smaller than the largest diameter of the cone tip 1023, but its hardness is lower enough to allow the cone tip 1023 to squeeze through its passage. After squeezing through the inner sleeve portion 1062, the cone tip 1023 can be blocked from sliding back out of the inner sleeve portion 1062 because its largest diameter is larger than the passage diameter of the inner sleeve portion 1062. Accordingly, the whole active device 100 can be reliably fastened into a desired portion of animal body by squeezing the pin 102 through the inner sleeve portion 1062.

A window 1064 is formed below the primary sleeve 1062 so as to allow sensors installed in the monitoring unit 108 to measure the vital signs such as body temperature.

By using a bonding technology, the base 106 and the cover 110 can be joined together so as to accommodate the processing unit 108 therein, in a waterproof manner. The processing unit 108 will be described in detail, below.

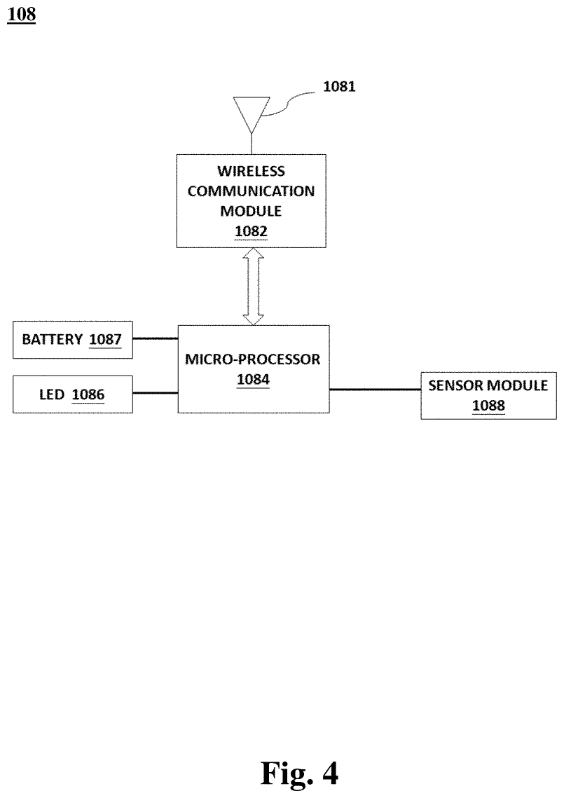

FIG. 4 is a block diagram illustrating the monitoring unit 108 according to embodiments of the invention. As shown in FIG. 4, the monitoring unit 108 includes a wireless communication module 1082, a micro-processor 1084, a light emitting diode (LED) 1086, a sensor module 1088, an antenna, etc. These components are mounted on a printed circuit board (PCB). Preferably, the PCB may be a double-sided type board such that various components may be installed on different sides based on the design.

The wireless communication module 1082 may communicate with an external apparatus such as gateway, via an antenna. The module 1082 may transmit data received from the external apparatus to the micro-processor 1084 and/or transmit data to the external under control of the micro-processor 1084. The wireless communication module 1082 may be configured to communicate using at least one wireless communication standard including Lora, Zigbee, etc.

The sensor module 1088 may include at least one kind of sensor, like temperature sensor, heart rate sensor, accelerator sensor, blood pressure sensor or any other sensor. According to embodiments of the invention, the sensor module 1088 is a temperature sensor, which is arranged to face the window 1064 so as to measure the body temperature of the animal in a non-contact manner. Measured results of the sensor module 1088 may be input to the micro-processor 1084.

The micro-processor 1084 can operate based on data input from the sensor module 1088 and can cause the communication module 1082 to transfer the measured vital signs like body temperature to the external apparatus such as gateway. Then, the gateway may send those data to a cloud system or any computing platform for management. The micro-processor 1084 may decide whether there is any abnormal event like fever or hypothermia, based on the measured data.

The LED indicator 1086 may indicates a status of the device 100, for example, the battery status, a system status, and so on. The micro-processor 1084 may detect the voltage of the battery powering the device, and cause the LED 1086 to indicate low power if the detected voltage is below a predetermined value.

In the foregoing, structures, various components and functions of the active device for monitoring animal of the invention have been described. According to this invention, the active device for monitoring animal can be easily and reliably fastened into ears of animals, automatically and accurately measure vital signs like body temperature of animals, and actively report the measured data, resulting in elimination or reduction of mass infection or mass mortality of fed animals, and considerable reduction of feeding cost. Although the active device is used for animals in the livestock farm in the aforementioned embodiment, it should be understood that the active device of the present invention is not limited to be used for animals in the livestock farm, it can also be used for animals such as pets, poultry, animals in the zoo or even wild animals.

While specific embodiments have been described, it should be understood by those skilled in the art that various changes in form and detail may be made therein without departing from the spirit and scope of this invention as defined by the appended claims. The scope is thus indicated by the appended claims and all changes which come within the meaning and range of equivalency of the claims are therefore intended to be embraced.

What is claimed is:

1. An active device for monitoring an animal, capable of measuring vital signs of the animal and actively signaling an external apparatus, the device comprising:
a pin adapted to penetrate a predetermined portion of the animal;
a gasket having two ring portions formed as one piece;
a base having a first sleeve portion for allowing the pin to squeeze through so as to be engaged with each other and blocking the pin from slipping off after engagement;
a monitoring unit including:
a sensor module for measuring vital signs of the animal;
a wireless communication module adapted to communicate with the external apparatus;
a controller adapted to receive measured data from the sensor module, and to control the wireless communication module to transmit the measured data to the external apparatus; and
a battery for powering the active device; and
a cover having a second sleeve portion to receive the first sleeve portion, and being joined with the base in a waterproof manner so as to accommodate the monitoring unit therein.

2. The device according to claim 1, wherein the sensor module comprises at least one sensor selected from a temperature sensor, a blood pressure sensor, a heart rate sensor, and an accelerator sensor.

3. The device according to claim 1, wherein the wireless communication module uses at least one of Lora, or Zigbee wireless communication standards to communicate with the external apparatus.

4. The device according to claim 1, wherein a hardness of the first sleeve portion is lower than that of the second sleeve portion.

5. The device according to claim 1, wherein the predetermined portion of the animal is an ear.

6. The device according to claim 5, wherein the sensor module is a temperature sensor module.

7. The device according to claim 6, wherein the controller determine whether the body temperature of the animal is abnormal based on the measured body temperature by the temperature sensor module.

8. The device according to claim 1, wherein the animal is a livestock.

9. The device according to claim 1, wherein the monitoring unit further comprises an LED indicator adapted to display a status of the device, and when the controller detects that electricity of the battery is low, the LED indicator is caused to indicate a low power status.

10. The device according to claim 2, wherein the monitoring unit further comprises an LED indicator adapted to display a status of the device, and when the controller detects that electricity of the battery is low, the LED indicator is caused to indicate a low power status.

11. The device according to claim 3, wherein the monitoring unit further comprises an LED indicator adapted to display a status of the device, and when the controller detects that electricity of the battery is low, the LED indicator is caused to indicate a low power status.

12. The device according to claim 4, wherein the monitoring unit further comprises an LED indicator adapted to display a status of the device, and when the controller detects that electricity of the battery is low, the LED indicator is caused to indicate a low power status.

13. The device according to claim 5, wherein the monitoring unit further comprises an LED indicator adapted to display a status of the device, and when the controller detects that electricity of the battery is low, the LED indicator is caused to indicate a low power status.

14. The device according to claim 6, wherein the monitoring unit further comprises an LED indicator adapted to display a status of the device, and when the controller detects that electricity of the battery is low, the LED indicator is caused to indicate a low power status.

15. The device according to claim 7, wherein the monitoring unit further comprises an LED indicator adapted to display a status of the device, and when the controller detects that electricity of the battery is low, the LED indicator is caused to indicate a low power status.

16. The device according to claim 8, wherein the monitoring unit further comprises an LED indicator adapted to display a status of the device, and when the controller detects that electricity of the battery is low, the LED indicator is caused to indicate a low power status.
